(19) United States
(12) Patent Application Publication
(10) Pub. No.: US 2016/0296104 A1
(43) Pub. Date: Oct. 13, 2016

(54) Biocompatible Sheath for Optical Device

(71) Applicant: Rememdia LC

(72) Inventor: Fraser Smith, Salt Lake City, UT (US)

(21) Appl. No.: 14/684,264

(22) Filed: Apr. 10, 2015

(51) Int. Cl. A61B 1/00; A61B 1/05

(52) U.S. Cl. CPC A61B 1/00144; A61B 1/00197; A61B 1/05

(57) ABSTRACT

An elongate medical device is disclosed which is configured for placement into a cavity of a body. The device has an elongate body member with a proximal end and a distal end, the body member having a light source, an image sensor, and a lens disposed about the distal end of the body, the lens being coupled to the image sensor. A disposable compliant sheath is removably disposed about the exterior of the elongate body member enclosing the elongate body member. The disposable sheath has a transparent optical member that is optically aligned with the lens.

### FIELD OF THE TECHNOLOGY

The present technology relates to improved devices, methods, and systems for medical devices. More particularly, the present technology relates to tools for preventing contamination of re-usable devices that are inserted into cavities of the body.

### BACKGROUND OF THE TECHNOLOGY AND RELATED ART

Optical instruments (e.g., endoscopes and the like) are used to visually examine internal organs or cavities within the human body to diagnose and/or treat various medical conditions. Endoscopes and their accessories (e.g., irrigation tubing) need to be reprocessed (cleaned and disinfected or sterilized) between each patient use. Failure to reprocess, or inadequate reprocessing of endoscopes and accessories, places patients at risk of exposure to various pathogens. Much of the literature on infection prevention in endoscopy identifies failure to follow established cleaning and disinfection/sterilization processes and use of damaged or malfunctioning reprocessing equipment or endoscopes as the leading causes of cross contamination. Endoscope reprocessing typically involves a six-step protocol that includes pre-cleaning, leak testing, manual cleaning, high-level disinfecting or sterilizing, rinsing and drying, and endoscope storing. A breakdown in any one of these steps could compromise the integrity of the process leading to an endoscopy-related contamination risk. Even following these procedures, risk of exposure is inherent in the reuse of any device that is repeatedly inserted into body cavities. This risk can result in transmission of infectious agents (e.g., hepatitis C, HIV, mycobacterium tuberculosis) and potentially lead to patient injury or death. Often in these cases, large numbers of patients are affected and must be notified about exposure to potentially contaminated endoscopic equipment.

### SUMMARY OF THE INVENTION

In light of the problems and deficiencies inherent in the prior art, the present invention seeks to overcome these by providing methods, devices, and systems for an elongate medical device configured for placement into a cavity of a body, comprising an elongate body member having a proximal end and a distal end. The body member has a light source, an image sensor, and a lens disposed about the distal end of the body. A disposable compliant sheath is removably disposed about the exterior of the elongate body member enclosing the elongate body member. The disposable sheath comprises a transparent optical member that is optically aligned with the lens.

In accordance with one aspect of the technology, an elongate medical device configured for placement into a cavity of a body is disclosed comprising an elongate body member having a proximal end and a distal end. The body member comprises a light source, an image sensor, and a lens disposed about the distal end of the body. The lens is optically coupled to the image sensor. A disposable compliant sheath is removably disposed about the exterior of the elongate body member enclosing the elongate body member. The disposable sheath comprises an optical member aligned with the lens. A first alignment member is disposed about the exterior of the elongate body member corresponding to a second alignment member disposed about the interior of the disposable sheath.

A method for facilitating the prevention of cross-contamination between successive medical procedures in which a medical device is used to image one or more body cavities is disclosed. The method can comprise providing a disposable compliant sheath comprising an optical member. The method can further comprise facilitating the removable disposition of the compliant sheath about the exterior of an elongate medical device configured for imaging a body cavity, wherein the compliant sheath can be configured to enclose at least a portion of the elongate medical device. The method can further comprise configuring the compliant sheath with an optical member configured to be optically aligned with a lens of the elongate medical device. The method further comprises facilitating the removal of the compliant sheath from the elongate medical device following the imaging procedure, wherein the compliant sheath may be discarded and a new one used with the elongate medical device.

A method of imaging a cavity of a patient is disclosed comprising placing an elongate optical medical device within the cavity of a patient, the elongate medical device comprising (i) an elongate body member having a proximal end and a distal end and further comprising a light source, an image sensor, and a lens disposed about the distal end of the body, and (ii) a disposable compliant sheath removably disposed about the exterior of the elongate body member enclosing the elongate body member, the disposable sheath comprising an optical member optically aligned with the lens. The method further comprises advancing the elongate optical medical device within the cavity of the patient to a desired target and acquiring an image of the desired target with the elongate optical medical device.

### BRIEF DESCRIPTION OF THE DRAWINGS

The present technology will become more fully apparent from the following description and appended claims, taken in conjunction with the accompanying drawings. Understanding that these drawings merely depict exemplary aspects of the present technology they are, therefore, not to be considered limiting of its scope. It will be readily appreciated that the components of the present technology, as generally described and illustrated in the figures herein, could be arranged and designed in a wide variety of different configurations. Nonetheless, the technology will be described and explained with additional specificity and detail through the use of the accompanying drawings in which:

FIG. 1 is a perspective view of an end of a medical device in accordance with one aspect of the present technology;

FIG. 2 is a perspective view of an end of a medical device in accordance with one aspect of the technology;

FIG. 3 is a side view of an end of a medical device in accordance with one aspect of the technology;

FIG. 4 is a side view of a sheath and optical member in accordance with one aspect of the technology;

FIG. 5a is a front view of an optical member in accordance with one aspect of the technology;

FIG. 5b is a front view of an optical member in accordance with one aspect of the technology;

FIG. 5c is a front view of an optical member in accordance with one aspect of the technology;

FIG. 6 is a perspective view of a medical device in accordance with one aspect of the technology; and

FIG. 7 is a side view of a sheath and optical member in accordance with one aspect of the technology.

### DETAILED DESCRIPTION OF EXEMPLARY ASPECTS OF THE TECHNOLOGY

The following detailed description of exemplary aspects of the technology makes reference to the accompanying drawings, which form a part hereof and in which are shown, by way of illustration, exemplary aspects in which the technology may be practiced. While these exemplary aspects are described in sufficient detail to enable those skilled in the art to practice the technology, it should be understood that other aspects may be realized and that various changes to the technology may be made without departing from the spirit and scope of the present technology. Thus, the following more detailed description of the aspects of the present technology is not intended to limit the scope of the technology, as claimed, but is presented for purposes of illustration only and not limitation to describe the features and characteristics of the present technology, to set forth the best mode of operation of the technology, and to sufficiently enable one skilled in the art to practice the technology. Accordingly, the scope of the present technology is to be defined solely by the appended claims. The following detailed description and exemplary aspects of the technology will be best understood by reference to the accompanying drawings, wherein the elements and features of the technology are designated by numerals throughout.

As used in this specification and the appended claims, the singular forms “a,” “an” and “the” include plural referents unless the context clearly dictates otherwise. Thus, for example, reference to “a layer” includes a plurality of such layers.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in an electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.”

An “SSID,” “solid state imaging device,” “SSID chip,” or “solid state imaging chip” in the exemplary embodiments generally comprises an imaging array or pixel array for gathering image data. In one embodiment, the SSID can comprise a silicon or silicon-like substrate or amorphous silicon thin film transistors (TFT) having features typically manufactured therein. Features can include the imaging array, conductive pads, metal traces, circuitry, etc. Other integrated circuit components can also be present for desired applications. However, it is not required that all of these components be present, as long as there is a means of gathering visual or photon data, and a means of sending that data to provide a visual image or image reconstruction.

The term “umbilical” can include the collection of utilities that operate the SSID or the micro-camera as a whole. Typically, an umbilical includes a conductive line, such as electrical wire(s) or other conductors, for providing power, ground, clock signal, and output signal with respect to the SSID, though not all of these are strictly required. For example, ground can be provided by another means than through an electrical wire, e.g., to a camera housing. The umbilical can also include other utilities such as a light source, temperature sensors, force sensors, fluid irrigation or aspiration members, pressure sensors, fiber optics, microforceps, material retrieval tools, drug delivery devices, radiation emitting devices, laser diodes, electric cauterizers, and electric stimulators, for example. Other utilities will also be apparent to those skilled in the art and are thus comprehended by this disclosure.

“GRIN lens” or “GRadient refractive INdex lens” refers to a specialized lens that has a refractive index that is varied radially from a center optical axis to the outer diameter of the lens. In one embodiment, such a lens can be configured in a cylindrical shape, with the optical axis extending from a first flat end to a second flat end. Thus, because of the differing refractive index in a radial direction from the optical axis, a lens of this shape can simulate the effects of a more traditionally shaped lens.

The directional terms “proximal” and “distal” are used herein to refer to opposite locations on a medical device. The proximal end of a device is defined as the end closest to the practitioner when the device is being used or manipulated by a practitioner. The distal end is the end opposite the proximal end, along the longitudinal direction of the device, or the end furthest from the practitioner. It is understood that, as used in the art, these terms may have different meanings with regard to devices deployed within the body of a patient (i.e., the “proximal” end may refer to the end closest to the head or heart of the patient depending on the application). For consistency, as used herein, the ends labeled “proximal” and “distal” prior to deployment remain the same regardless of whether the device is disposed within a patient.

“Lens” as used herein means a transmissive optical device which affects the focusing of a light beam through refraction. The lens may comprise a single piece of material or several materials disposed along a common axis.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

An initial overview of technology is provided below and specific technology is then described in further detail. This initial summary is intended to aid readers in understanding the technology more quickly, but is not intended to identify key or essential features of the technology, nor is it intended to limit the scope of the claimed subject matter.

Aspects of the current technology are intended to provide improved systems, devices, and methods for repeatedly using an optical instrument, such as an endoscope, within a cavity or vessel of a patient while minimizing the likelihood of contamination from one patient to another. Broadly speaking, aspects of the technology are embodied in a compliant sheath that is removably disposed about the exterior of an elongate medical device. The compliant sheath comprises a transparent optical medium disposed about one end of the sheath. The sheath is used in connection with an elongate medical device configured for placement into a cavity of a body. In one aspect, the elongate medical device comprises an elongate body member having a proximate end and a distal end, the body member comprising a light source, an image sensor, and a first lens disposed about the distal end of the body. The first lens is coupled to the image sensor and operates in connection with a processor and a display device to image cavities and/or vessels within the body of the patient. Prior to inserting the elongate body member into the patient, the compliant sheath with the optical medium is removably disposed about the exterior of the elongate body member, enclosing the elongate body member. Once a procedure is completed on one patient, the compliant sheath is removed from the elongate body member and disposed of. When the practitioner desires to use the medical device again, a new disposable sheath is placed about the exterior of the elongate body member. Advantageously, problems associated with improper or inadequate cleaning procedures are mitigated by virtue of the fact that the exterior of the elongate body member never comes into contact with any fluids within the patient permitting repeated use of expensive imaging devices while minimizing the risk of exposure.

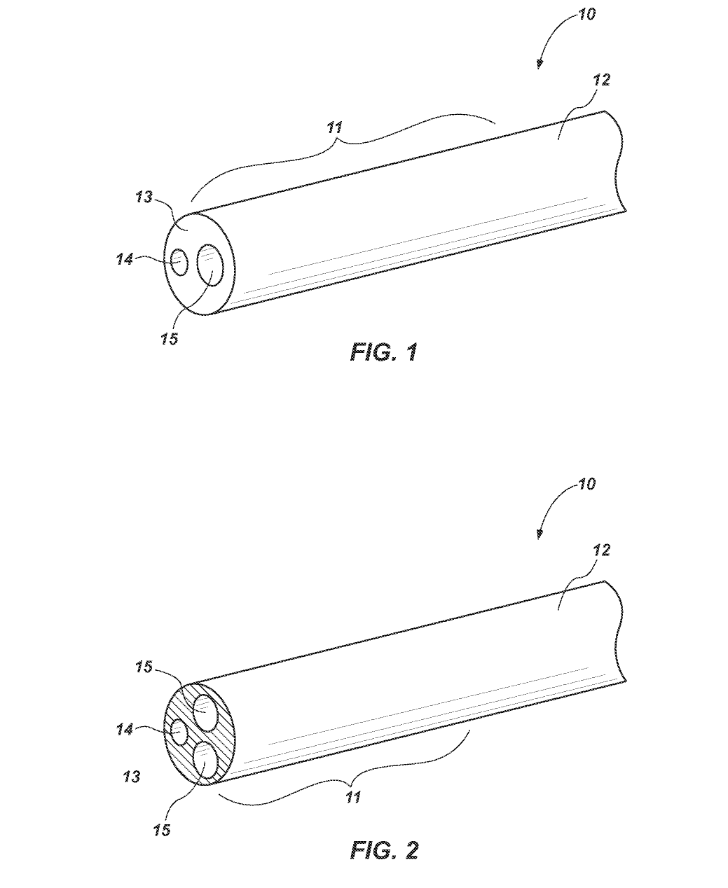

With reference to FIGS. 1-4, an example medical device 10 is disclosed in accordance with one aspect of the technology. The distal end 11 of an elongate body 12 of the medical device 10 comprises a front flat face 13 and includes a light source 14 and one or more lenses 15. The lens 15 is operatively coupled to an image sensor 16. The elongate body 12 has a continuous outer diameter from a distal end 11 of the elongate body 12 to a proximal end. The image sensor converts an image input as light from the target into an electrical signal and transmits the electrical signal to a digital processing device. Non-limiting examples of an image sensor include a charge coupled device (CCD) and/or a complementary metal oxide semiconductor (CMOS). The CCD image sensor includes a photodiode, a CCD, and a signal detection circuit, which are formed over a P-type impurity layer. The photodiode serves to convert light incident from outside into an electric charge, the CCD serves to transmit the electric charge to the signal detection circuit, and the signal detection circuit serves to convert the electric charge into a voltage. The CMOS image sensor includes a CMOS transistor configured to convert an input image into an electrical signal. The image sensor is operatively coupled to an umbilical which powers the image sensor and the light source. The umbilical is coupled to a processor and display for displaying an image of the area within the field-of-view of the image sensor. The light source 14 can include, in various aspects of the technology, semiconductor sources (light emitting diodes (LED)), edge emitting diodes (ELED), superluminscent diodes (SLD), mode-lock lasers (e.g. TiAl2O3, Cr:Mg2SiO4, CrLiSrAlF6), fiber optics, rare earth doped fibers (REDF) (Yb, Nd, Er, Pr, Tm), super-continuum or Raman sources, and others as recognized by those skilled in the art.

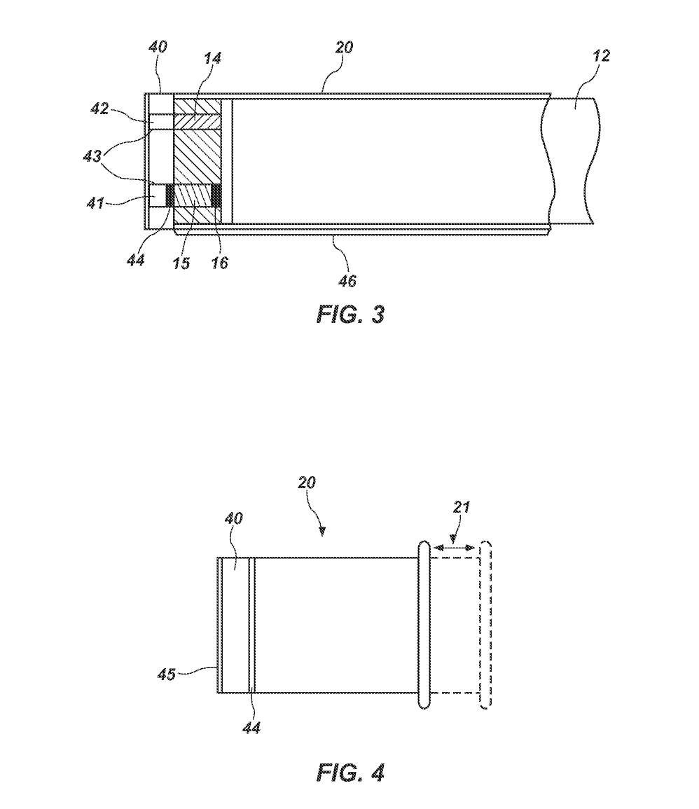

With reference to FIGS. 3 and 4, and generally to FIGS. 1 and 2, in accordance with one aspect of the technology, a compliant (i.e., non-rigid) sheath 20 is further disclosed. The compliant sheath 20 is elongate and is shaped to approximate the elongate body 12 of the medical device 10. In one aspect of the technology, the compliant sheath 20 may be constructed from any suitable biocompatible material. In one example, the sheath 20 can be comprised of a polymeric material. In another example, the sheath 20 can be comprised of an elastomeric material. Suitable elastomeric materials include, but are not limited to, polyurethane-based elastomer, polyester-based elastomer, polyolefin-based elastomer, polyamide-based elastomer, polystyrene-based elastomer, fluorine-based elastomer, silicone rubber, fluororubber, and latex rubber. In another example, the sheath 20 may be comprised of a substantially inelastic material. In one aspect, the sheath 20 can be comprised of an inelastic material such that length of the sheath 20 does not change substantially, yet is compliant enough to bend with the elongate body 12 as it is advanced through pathways within the body of the patient. In one specific aspect, the sheath 20 can comprise expanded polytetrafluoroethylene (ePTFE). In another aspect of the technology, the sheath 20 can include directionally oriented fibers (e.g., SPECTRA or KEVLAR fibers, or others as recognized by those skilled in the art) such as longitudinally oriented fibers, or polymer reinforced fibers to improve rigidity yet permit compliance during navigation of the elongate optical device 10 within the body of the patient. In accordance with one aspect of the technology, the sheath 20 is molded or extruded as a single unit with no seams in order to minimize the likelihood of sheath 20 failure during a procedure.

In additional aspects, the sheath 20 can be coated with one or more materials as needed. For example, the sheath 20 can be coated with a hydrophilic or lubricous material to facilitate advancement of the medical device 10 and sheath 20 through the patient anatomy. In another aspect, or in addition, the sheath 20 (both inside surfaces and outside surfaces; inside surface or outside surface) can be coated with anticoagulants, such as heparin, antibiotics and plaque formation inhibitors, or other materials as suits a particular purpose.

In one aspect of the technology, an inner diameter of the sheath 20 is about the same as the outer diameter of the elongate body 12. The wall thickness of the sheath 20 can range from approximately 0.1 mm to 1 mm. While FIG. 3 illustrates a gap between the outer wall of elongate body 12 and the inner wall of sheath 20, the gap is intended to illustrate a distinction between the elongate body 12 and the sheath 20. In accordance with one aspect of the technology, the sheath 20 is in direct contact with the elongate body 12. In this manner, the overall width of the medical device 10 being inserted into the cavity of the patient is minimized. In one aspect of the technology, the sheath 20 is configured to be placed about the exterior of the elongate body 12 and removed from the elongate body 12 by rolling the sheath 20 on and/or off of the elongate body 12 as shown in FIG. 4 at 21. In yet another aspect of the technology, the compliant sheath 20 can comprise a secondary lumen disposed about an exterior of the sheath 20 about the longitudinal axis of the sheath 20. The secondary lumen can be used to be placed over a guide-wire for navigating the medical device 10 within cavities and/or vessels of the patient and/or placement of other tools (e.g., cauterizers, forceps, etc.) at the distal end of the medical device.

In one aspect of the technology, the compliant sheath 20 comprises a non-compliant (i.e., rigid) optical member 40. The optical member 40 can comprise a flat back-end configured to be operative with (e.g., mate, interface, etc.) the front face 13 of the medical device 10. In one sense, the optical member 40 can comprise a transparent optical member configured to be positioned about, near or on the lens 15 (be it on the front 13 or side of the distal end 11 of the medical device 10) in such a way as to minimize image distortion through the optical member 40 to the lens 15. While it is anticipated that in some embodiments the back end of optical member 40 will be in contact with the front end 13 of the elongate member 12, in other embodiments the optical member 40 may not necessarily be in contact with the elongate member 12 (either completely or partially). For example, a transparent optical gel or fluid can be placed on the distal end 11 of elongate member 12 to minimize pockets of air between the front end 13 of the elongate member 12 and the back end of the optical member 40. In accordance with an additional aspect of the technology, the back end of optical member 40 can comprise a compliant compartment filled with a transparent optical gel or fluid intended to conform to fill any air gaps between the two adjacent surfaces to minimize image distortion.

In one aspect of the technology, the optical member 40 comprises a lens 41 configured to focus an image about the lens 15 of medical device 10 and a light conducting element 42 configured to conduct light from light source 14 to a target within a cavity of the body. The light conducting element 42 and the lens 41 of the sheath 20 can be separated by an opaque film or wall 43 to minimize light pollution. More than one lens 41 can be disposed about the optical member 40 to accommodate more than one lens 15 and/or to propagate varying types of images (i.e., magnified, filtered, etc.) to the lens or lenses 15. In addition, it is not necessary that all parts of the optical member 40 be transparent. Rather, in accordance with one aspect, only the regions within conducting element 42 and lens 41 are transparent. The remaining portions of optical member 40 can be substantially opaque. The optical member 40 can be made generally from thermoplastics including polycarbonates, polyesters or other materials such as silicone, glass or other materials known in the art. In accordance with one aspect of the technology, the optical member 40 can be integrally formed with the sheath 20. In another aspect, the optical member 40 can be formed separate from the sheath 20 and secured to the sheath 20 prior to being placed on the elongate body 12. The optical member 40 and/or its attendant lens(es) 41 may be a GRIN lens or other type of lens and may be biconvex, biconcave, plano-convex, plano-concave, or convex-concave as suits a particular purpose. In addition, the optical member 40 and/or lenses 41 can be equi-convex or equi-concave as suits a particular purpose.

With reference now to FIG. 4, one or more additional elements can be associated with the optical member 40. In one non-limiting example, an optical filter media 44 can be disposed behind optical member 40 and can be configured to modify the light from a target entering the optical member 40. The filter media 44 can be a linear or non-linear filter as suits a particular purpose. In one aspect, the filter media 44 selectively transmits light of different wavelengths. In one aspect, the filter media 44 can be an absorptive filter. In another aspect, the filter 44 can comprise an interference or dichroic filter. Other filters are contemplated for use herein as suits a particular purpose, and as will be apparent to those skilled in the art.

In yet another aspect of the technology, the front surface of the optical member 40 can comprise a layer 45 intended to scatter light emanating from light source 14. The layer 45 can comprise a nano-particle sized metal (e.g., gold, silver, or copper, etc.) (or other metal layer) deposited about the front surface of optical member 40 in front of the area aligned with light source 14. The layer 45 scatters light for use in connection with Raman spectroscopy. Raman spectroscopy is a spectroscopic technique used to observe vibrational, rotational, and other low-frequency modes in a system. The process relies on inelastic scattering, or Raman scattering, of monochromatic light, usually from a laser in the visible, near infrared, or near ultraviolet range. The laser light interacts with molecular vibrations, photons or other excitations in the system, resulting in the energy of the laser photons being shifted up or down. The shift in energy gives information about the vibrational modes in the system. Infrared spectroscopy yields similar, but complementary, information. A sample or target is illuminated with a laser beam. Electromagnetic radiation from the illuminated spot is collected with a lens and sent through a monochromator. Elastic scattered radiation at the wavelength corresponding to the laser line (Rayleigh scattering) is filtered out, while the rest of the collected light is dispersed onto a detector by either a notch filter or a band pass filter. The unfiltered media is processed by a computer processor and presented on a display device.

In one aspect, the beam from light source 14 is focused upon the target to generate in-elastically scattered radiation, which is optically collected and directed into lens 15 via lens 41, for example, and a wavelength-dispersive spectrometer in which a detector converts the energy of impinging photons to electrical signal intensity. Advantageously, the layer 45 enhances imaging of certain aspects of targets within the patient. The layer 45 can be disposed across the entire surface of the optical member 40 or only part of the surface of optical member 40 (e.g., light conducting element 42) as suits a particular purpose. In accordance with one aspect of the technology, the distal end of the optical element 40 and layer 45 are placed in direct contact with the target within the patient prior to propagating the beam of light onto the target.

In accordance with one aspect of the technology, the layer 45 can comprise a composite nanostructure that includes, but is not limited to, a core, a reporter molecule, and an encapsulant material. The reporter molecules are disposed (bonded) onto the core, while the encapsulant material covers and protects the core and reporter molecules. The core optically enhances the imaging spectrum, while the reporter molecule provides a distinct spectroscopic imaging signature. Disposing the encapsulant material over the core and reporter molecule does not substantially impact the spectroscopic imaging signature of the reporter molecule, while protecting the core and reporter molecules. The core can be made of materials such as, but not limited to, metals. In particular, the core can be made of materials such as, but not limited to, gold, silver, copper, transition metals (e.g., Zn, Ni, and Cd), semiconductors (e.g., CdSe, CdS, and InAs), and combinations thereof.

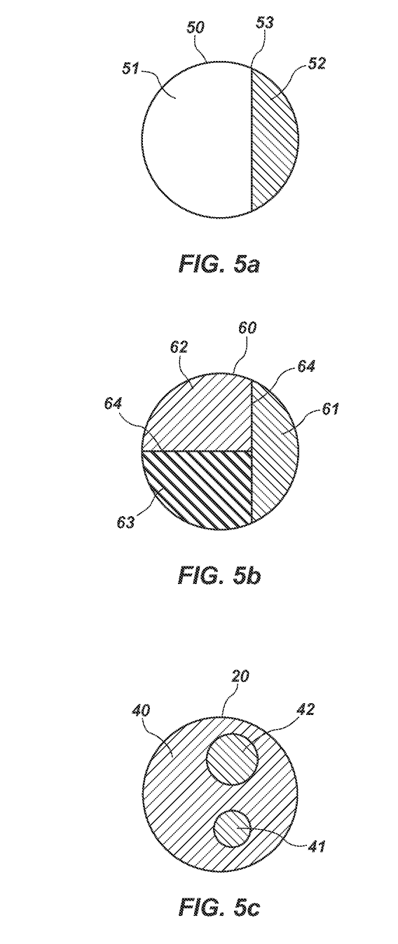

With reference generally to FIGS. 1-2 and 5a through 5c, in accordance with one aspect of the technology, the light conducting element 42 and the lens 41 can be sized to approximate the opening of the light source 14 and the lens 15 of the elongate body 12, respectively such as that shown on FIG. 5c. In this manner, existing medical devices (such as endoscopes) that require extensive cleaning and treatment between uses can be retrofit with a disposable sheath that matches the properties of the medical device while minimizing the likelihood of exposure through cross-contamination. In another aspect of the technology, the optical member 40 can be divided into larger “generic” sections devoted to different purposes without the need to be sized to the dimensions of a specific device. For example, FIG. 5a illustrates a front view of a transparent optical member 50 divided into a first section 51 and a second section 52. The first section 51 is sized to be placed about the front end 13 of the elongate body 12 and positioned such that the lens 15 of the elongate body 12 is located somewhere within the region of the first section 51. In one aspect, the first section 51 can comprise a focusing lens. In another aspect, it can comprise a magnifying lens. In this aspect, the second section 52 can be positioned about the front face 13 of the elongate body 12 such that the light source 14 is located within the region of the second section 52. An opaque wall 53 can be located between the first 51 and second sections 52 to minimize light pollution from the light source 14 into the lens 15. In this manner, numerous types and styles of medical devices can be fit with a single style of disposable sheath 20. With reference to FIG. 5b, in another aspect of the technology, a transparent optical member 60 can be divided into three sections 61, 62, 63 with an opaque wall 64 between each section. In one aspect, the first and second sections 61, 62 correspond to first and second lenses (e.g., the lenses 15 on FIG. 2) associated with the elongate body 12. Each of the first and second sections (61, 62) can comprise a focusing lens, a magnifying lens, or other lens as suits a particular application. In this aspect, section 63 corresponds to a light source 14 and is intended to pass light through the optical member 60 onto a target for imaging. In one aspect, the light source 14 is disposed outside of the image plane lenses associated with the optical member 60.

In aspects of the present technology, the optical members 40, 50, 60, for example, can have a uniform thickness. However, in other aspects of the technology, different sections of the optical members 40, 50, 60 can have varying thicknesses as suits a particular application. In one non-limiting example, with reference to FIG. 5a, the first section 51 is thicker than the second section 52. The second section 52, configured to permit light to pass through to a target to be imaged, is thinner than the first section 51. Other configurations are contemplated for use herein as suits a particular purpose.

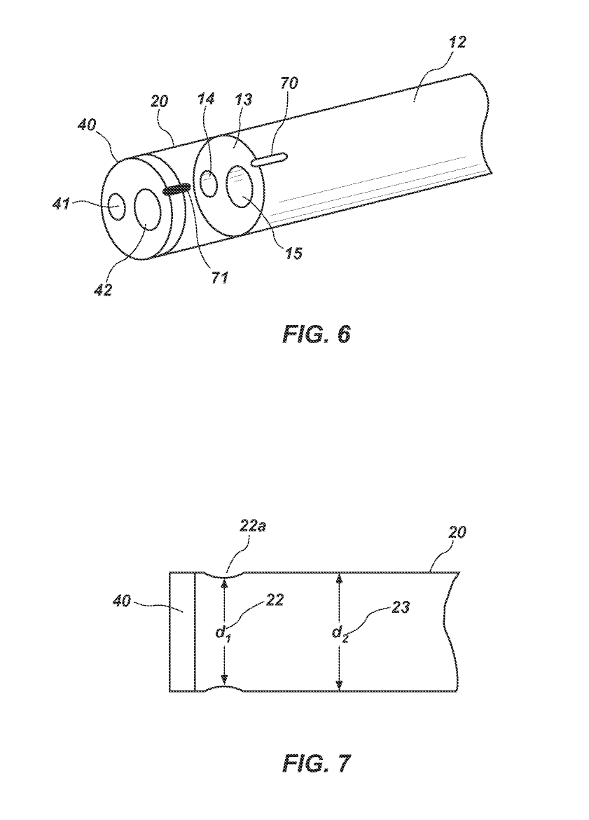

With reference generally to FIG. 6, in order to accommodate proper alignment of lens 41 with lens 15 and the light conducting element (or window) 41 with the light source 14, a groove 70 can be disposed, formed, etc. about a side surface of elongate body 12 corresponding to a mating tab 71 disposed, formed about the sheath 20. The groove 70 and tab 71 can be configured such that when the two are mated together window 41 is axially aligned with light source 14 and lens 41 is axially aligned with lens 15. As such, the groove 70 and tab 71 can comprise alignment members facilitating proper alignment between the sheath 20 and elongate body 12. In addition, the groove 70 and tab 71 can provide structural stability to the optical member 40 as it rests on the distal end 11 of the elongate body 12. In this manner, movement of the optical member 40 during operation of the medical device 10 can be minimized. In other embodiments, a color mark on the outside of the optical member 40 corresponding to tab 71 can be provided with a corresponding color mark on the elongate body 12 corresponding to the groove 70 to assist in alignment of the optical member 40 about the elongate body 12. In still other aspects, there can be no groove or tab assembly, rather the alignment can be dictated solely by color marks associated with the respective pieces and/or other visible indicia of alignment. Likewise, use of mating tab 71 and groove 70 can be employed without use of the color marks or other visible indicia of alignment. Still other types of alignment members, indicia, etc. can be implemented as will be recognized by those skilled in the art.

With reference to FIG. 7, in accordance with one aspect of the technology, the sheath 20 can comprise a first diameter 22 near a distal end 11 of the elongate body 12 and a second diameter 23, such as one corresponding to another part of the sheath 20 or to the remainder of the sheath 20. In one aspect, the first diameter 22 can be less than the second diameter 23. The second diameter 23 can be sized less than the outer diameter of the elongate body 12 whereas the second diameter 23 can be sized about equal to the outer diameter of the elongate body 12. The sheath 20 can comprise a resilient material near the area of the first diameter 22 which can be biased in a “narrowed” configuration shown generally at 22a. When the sheath 20 is placed on the elongate body 12, the narrowed area 22a of the sheath 20 can be forced into an open configuration. The resilient characteristics of the sheath 20 about area 22a can create an interference fit for the sheath 20 about the distal end 11 of the elongate body 12. While the narrowed configuration is disclosed in one location in FIG. 7, it is understood that the narrowed configuration can be placed at various locations about the sheath 20 and in various orientations and various lengths as suits a particular purpose. For example, in one aspect where the elongate body 12 comprises a light source 14 and lens 15 disposed laterally with respect to a longitudinal axis of the elongate body 12 (e.g., about a side surface of the elongate body 12), the sheath 20 can be narrowed on either side of the light source 14 and lens 15 to assist in the securement of the optical member 40 about the side of the elongate body 12.

In accordance with one aspect of the technology herein, a method for facilitating prevention of cross-contamination between successive medical procedures in which a medical device is used to image one or more body cavities is disclosed. The method can comprise providing a disposable compliant sheath comprising an optical member. The method can further comprise facilitating the removable disposition of the compliant sheath about the exterior of an elongate medical device configured for imaging a body cavity, wherein the compliant sheath can be configured to enclose at least a portion of the elongate medical device. The method can further comprise facilitating the removal of the compliant sheath from the elongate medical device following the imaging procedure, wherein the compliant sheath may be discarded and a new one used with the elongate medical device.

In one aspect, the elongate body can comprise an elongate body member having a proximal end and a distal end, a light source, an image sensor, and a lens disposed about the distal end of the body.

The method can further comprise configuring the compliant sheath with an optical member configured to be optically aligned with the lens of the elongate medical device.

In accordance with one aspect of the technology herein, a method of imaging a cavity of a patient is disclosed comprising placing an elongate optical medical device within the cavity of a patient as described herein. In one aspect, the elongate medical device comprises (i) an elongate body member having a proximal end and a distal end and further comprising a light source, an image sensor, and a lens disposed about the distal end of the body, and (ii) a disposable compliant sheath removably disposed about the exterior of the elongate body member enclosing the elongate body member, the disposable sheath comprising an optical member optically aligned with the lens. The method further comprises advancing the elongate optical medical device within the cavity of the patient to a desired target and acquiring an image of the desired target with the elongate optical medical device. Once the practitioner has completed imaging of desired targets within the body of the patient, the elongate optical medical device is removed from the cavity of the patient and the disposable compliant sheath is removed from the elongate optical medical device. The compliant sheath is then disposed of and a new sheath is placed on the elongate optical medical device.

The foregoing detailed description describes the technology with reference to specific exemplary aspects. However, it will be appreciated that various modifications and changes can be made without departing from the scope of the present technology as set forth in the appended claims. The detailed description and accompanying drawings are to be regarded as merely illustrative, rather than as restrictive, and all such modifications or changes, if any, are intended to fall within the scope of the present technology as described and set forth herein.

More specifically, while illustrative exemplary aspects of the technology have been described herein, the present technology is not limited to these aspects, but includes any and all aspects having modifications, omissions, combinations (e.g., of aspects across various aspects), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus-function are expressly recited in the description herein. Accordingly, the scope of the technology should be determined solely by the appended claims and their legal equivalents, rather than by the descriptions and examples given above.

1. An elongate medical device configured for placement into a cavity of a body, comprising:
an elongate body member having a proximal end and a distal end, the elongate body member comprising a light source, an image sensor, and a lens disposed about the distal end of the body; and
a disposable compliant sheath removably disposed about the exterior of the elongate body member at least partially enclosing the elongate body member, the disposable sheath comprising a transparent optical member optically aligned with the lens.

2. The elongate device of claim 1, wherein the lens disposed about the elongate body member is a first lens and wherein the optical member comprises a second lens axially aligned with the image sensor and the first lens.

3. The elongate device of claim 2, wherein the light path of the light source is outside the image plane of the second lens.

4. The elongate device of claim 2, wherein a front end of the first lens is disposed proximate to the back end of the second lens.

5. The elongate device of claim 1, wherein an inner and outer surface of the sheath comprises antibiotics.

6. The elongate device of claim 1, wherein the sheath comprises a plurality of fiber-reinforced polymers disposed about a longitudinal axis of the sheath.

7. The elongate medical device of claim 1, wherein the elongate body member comprises a substantially continuous outer diameter from a proximal end of the elongate body member to a distal end of the elongate body member.

8. The elongate device of claim 7, wherein the sheath comprises a first inner diameter in a proximal end of the sheath and a second inner diameter in a distal end of the sheath.

9. The elongate device of claim 8, wherein the first inner diameter is substantially equivalent to the outer diameter of elongate body member and the second inner diameter is less than the outer diameter of the elongate body member.

10. The elongate device of claim 2, further comprising a compartment comprising a transparent gel disposed between the front end of the first lens and the back end of the second lens.

11. The elongate device of claim 1, wherein the optical member comprises a first section and a second section, the first section being a magnifying lens and the second section being a non-magnifying lens.

12. The elongate device of claim 2, wherein a distal end of the second lens comprises a metal layer, wherein the metal layer is deposited about a front surface of the second lens.

13. The elongate device of claim 12, wherein the second lens comprises a first section and a second section, the first section being axially aligned with the first lens and the second section disposed in the light path of the light source, wherein only the second section of the second lens comprises the metal layer.

14. An elongate medical device configured for placement into a cavity of a body, comprising:
an elongate body member having a proximal end and a distal end, the body member comprising a light source, an image sensor, and a lens disposed about the distal end of the body;
a disposable compliant sheath removably disposed about the exterior of the elongate body member enclosing the elongate body member, the disposable sheath comprising a transparent optical member aligned with the lens;
a first alignment member disposed about the elongate body member that corresponds to a second alignment member disposed about the disposable sheath.

15. The elongate device of claim 14, wherein the optical member comprises a first section, a second section, and a third section, wherein the first section is a magnifying section, the second section is an image focusing section, and the third section is a transparent optical member.

16. The elongate device of claim 15, wherein the first section is separated from respective other sections by an opaque layer.

17. The elongate device of claim 14, further comprising an optical filter media disposed about the optical member.

18. The elongate device of claim 14, wherein the optical member is disposed about a side surface of the sheath.

19. A method of imaging a cavity of a patient, comprising:
placing an elongate optical medical device within the cavity of a patient, the elongate medical device comprising (i) an elongate body member having a proximal end and a distal end and further comprising a light source, an image sensor, and a lens disposed about the distal end of the body, and (ii) a disposable compliant sheath removably disposed about the exterior of the elongate body member enclosing the elongate body member, the disposable sheath comprising a transparent optical member optically aligned with the lens;
advancing the elongate optical medical device within the cavity of the patient to a desired target; and
acquiring an image of the desired target with the elongate optical medical device.

20. The method of claim 19, further comprising the steps of removing the elongate optical medical device from the cavity of the patient and thereafter removing the disposable compliant sheath from the elongate optical medical device.

21. A method for facilitating prevention of cross-contamination between successive medical procedures in which a medical device is used to image one or more body cavities, the method comprising:
providing a disposable compliant sheath comprising an optical member;
facilitating the removable disposition of the compliant sheath about the exterior of an elongate medical device configured for imaging a body cavity, wherein the compliant sheath can be configured to enclose at least a portion of the elongate medical device; and
configuring the compliant sheath with a transparent optical member configured to be optically aligned with a lens of the elongate medical device.

22. The method of claim 21, further comprising facilitating the removal of the compliant sheath from the elongate medical device following the imaging procedure, wherein the compliant sheath may be discarded and a new one used with the elongate medical device.

23. The method of claim 21, wherein at least a portion of an outside surface of the optical member comprises a metal layer.

24. The method of claim 23, further comprising propagating a monochromatic beam of light through the metal layer and onto a target, wherein the optical member is in direct contact with the target.

25. The method of claim 21, further comprising obtaining an array of Raman spectral data collected from the target and processing the Raman spectral data.
